(19) United States
(12) Patent Application Publication
(10) Pub. No.: US 2025/0090176 A1
(43) Pub. Date: Mar. 20, 2025

(54) System and Method for Creating Customized Spacers for Use in Knee Arthroplasty

(71) Applicant: Eugene Thomas Miller, Fort Washington, PA (US)

(72) Inventor: Eugene Thomas Miller, Fort Washington, PA (US)

(21) Appl. No.: 18/829,168

(22) Filed: Sep. 9, 2024

Related U.S. Application Data
(60) Provisional application No. 63/582,637, filed on Sep. 14, 2023.

(51) Int. Cl. A61B 17/15; A61B 17/17; A61F 2/38; A61F 2/46

(52) U.S. Cl. CPC A61B 17/155; A61B 17/157; A61B 17/1764; A61F 2/3859; A61F 2/389; A61F 2002/4615

(57) ABSTRACT

A system and method for customizing a knee prosthesis that has a femoral component, a tibial component, and a spacer construct. A gap space exists between the femoral component and the tibial component that is measured in vivo. A spacer blank is provided. The spacer blank is shaped into a spacer construct on-demand during the surgical procedure. The spacer construct that is produced matches the optimal size needed for the gap space that was measured. The spacer blank is shaped by being placed in a jig. Various tools are then used to cut features into the spacer blank, wherein the tools are guided by the jig. This produces the spacer construct of the proper size.

### RELATED APPLICATIONS

This application claims the benefit of U.S. Provisional Application No. 63/582,637, filed Sep. 14, 2023.

### BACKGROUND OF THE INVENTION

### 1. Field of the Invention

In general, the present invention relates to prosthetics and methods used during a knee arthroplasty procedure. More particularly, the present invention relates to the design and manufacture of the spacer used between the femoral component and the tibial component of the knee prosthesis.

### 2. Prior Art Description

Total knee replacement, also called knee arthroplasty, is a surgical procedure used to resurface a knee damaged by injury and/or disease. During a knee arthroplasty, the knee joint is replaced with a prosthetic joint. The prosthetic joint typically has a femoral component that attaches to the femur, a tibial component that attaches to the tibia, and a plastic spacer that attaches to the tibial component. The spacer serves as the wear surface, wherein the femoral component moves against the spacer. During a knee arthroplasty, damaged and/or diseased sections of the femur and the tibia are cut away. The amount of bone removed varies from person to person. For this reason and a few others, the gap space between the femoral component of the prosthesis and the tibial component of the prosthesis varies from person to person. One way to compensate for variations between patients is to control the amount of bone that is removed and use custom femoral and/or tibial components. Such prior art methodologies are exemplified in U.S. Pat. No. 10,912,658 to Jackson, and U.S. Patent Application Publication No. 2016/0242919 to Engh. However, the most common process used to compensate for gap spaces of different sizes between the femoral and tibial elements is to alter the size of the spacer.

In a knee arthroplasty, the surgeon installs the femoral element and the tibial element. A selection of different sized spacers is made available in the operating room. After the femoral and tibial components are installed, the surgeon selects the spacer that best fits the space present. The selection is only a best fit and not an optimal fit. Spacers are incremented in size. Consequently, one spacer is often too small while the next sixed spacer is too large. In addition, a surgeon typically has access to only one of each sized spacer during an operation. If the spacer is damaged or contaminated during the procedure, the surgeon has no choice but to use another spacer of a different and less optimal size. Since the size of the spacer is not optimized to the actual space between the femoral and tibial components, the prosthetic joint is compromised. This can significantly shorten the life of the prosthetic joint and/or cause pain and other complications to the patient.

A need therefore exists for a system that enables one or more spacers of any size to be created on-demand during a knee arthroplasty procedure. In this manner, the space available for the spacer can be measured and a spacer can be created that is optimized for the measured space. This need is met by the present invention as described and claimed below.

### SUMMARY OF THE INVENTION

The present invention is a system and method for customizing a knee prosthesis that has a femoral component, a tibial component, and a spacer construct. The femoral component and the tibial component of the knee prosthesis are installed during a surgical procedure. One installed, a gap space between the femoral component and the tibial component is measured in vivo. Knowing the size of the gap space, an optimal size for the spacer construct can be determined.

A spacer blank is provided that is larger than the optimal size for the spacer construct. The spacer blank is shaped into the spacer construct during the surgical procedure. As such, the spacer construct that is produced matches the optimal size needed for the gap space that was measured.

The spacer blank is shaped by being placed in a jig that receives and retains the spacer blank. In the jig, the spacer blank is cut to the proper dimensions. Various tools are then used to cut features into the spacer blank, wherein the tools are guided by the jig. This produces the spacer construct of the proper size. The spacer construct is then utilized in the knee replacement procedure. If the spacer construct becomes damaged or contaminated during the surgical procedure, another spacer construct can be fabricated on-demand.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 shows a knee joint containing an exemplary knee prosthesis;

FIG. 2 is an exploded view of the exemplary embodiment of FIG. 1;

FIG. 3 shows a front view of a spacer blank as it would appear before being worked, and a finished spacer construct made from the spacer blank;

FIG. 4 shows a retention base and a spacer blank used in the methodology for custom sizing a spacer construct;

FIG. 5 shows a retention base, a spacer blank, and a cutting jig used in the methodology for custom sizing a spacer construct;

FIG. 6 shows a retention base, a spacer blank, and a milling jig used in the methodology for custom sizing a spacer construct;

FIG. 7 shows a front view of a spacer blank as it would appear before being worked and finished spacer construct made from the spacer blank using an alternate method of construction.

### DETAILED DESCRIPTION OF THE DRAWINGS

Although the present invention system and method can be embodied in many ways, only a few exemplary embodiments are illustrated, The exemplary embodiments are being shown for the purposes of explanation and description. The exemplary embodiments are selected in order to set forth some of the best modes contemplated for the invention. The illustrated embodiments, however, are merely exemplary and should not be considered as limitations when interpreting the scope of the claims.

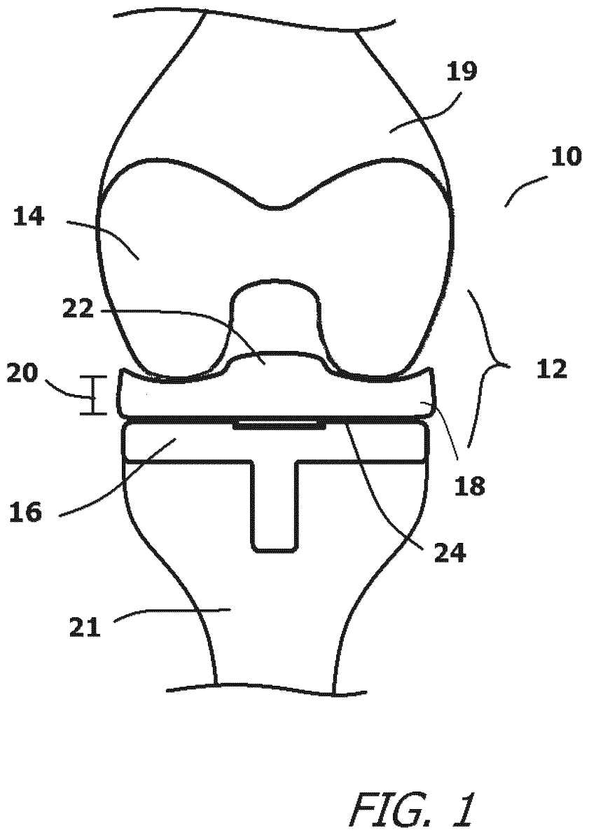

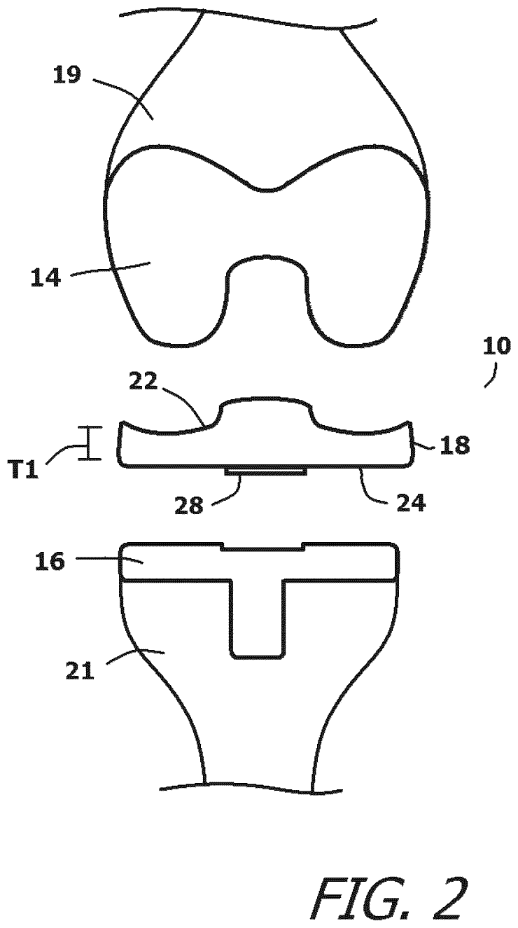

Referring to FIG. 1 and FIG. 2, a knee joint 10 containing a knee prosthesis 12 is shown. The knee prosthesis 12 includes a femoral component 14, a tibial component 16, and a spacer construct 18. The femoral component 14 is surgically affixed to the femur 19 using traditional techniques. The tibial component 16 is surgically affixed to the tibia 21 using traditional techniques. Once the femoral component 14 and the tibial component 16 are installed, a gap space 20 exists between the two components 14, 16. The size of the gap space 20 varies from patient to patient depending upon anatomy and how much bone is removed from the femur 19 and tibia 21. The spacer construct 18 is sized to optimally fit within that gap space 20 and enable the overall knee prosthesis 12 to function properly.

Once the spacer construct 18 is installed, the femoral component 14 rests upon the spacer construct 18 and wears against the spacer construct 18 when the knee joint 10 bends. The spacer construct 18 has a femoral-facing surface 22, a tibial-facing surface 24, and a primary body thickness T1 between the femoral-facing surface 22 and the tibial-facing surface 24. The femoral-facing surface 22 of the spacer construct 18 has precisely machined features 26 that enable the spacer construct 18 to properly interact with, and wear against, the femoral component 14. Likewise, the tibial-facing surface 24 of the spacer construct 18 has connection features 28 that enable the spacer construct 18 to precisely interconnect with the tibial component 16.

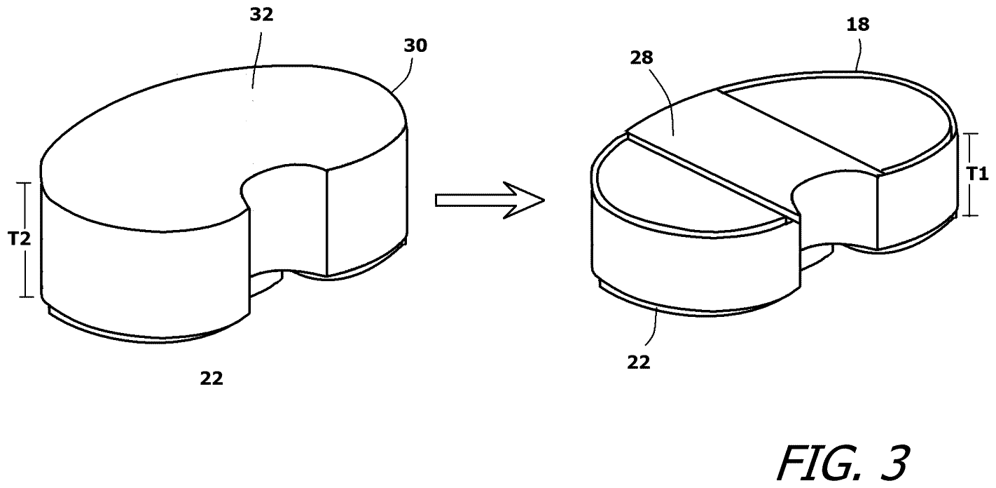

In order for the spacer construct 18 to be precisely fitted to the gap space 20 between the femoral component 14 and the tibial component 16, the spacer construct 18 is partially fabricated on-demand during the surgical procedure. Referring to FIG. 3 in conjunction with FIG. 1 and FIG. 2, it can be seen that a spacer blank 30 is provided. The spacer blank 30 is selected for the make and model of the knee prosthesis 12 being used by the surgeon. The spacer blank 30 is shown inverted with its a femoral-facing surface 22 facing down. The femoral-facing surface 22 is factory finished and is the same as will be part of the finished spacer construct 18. The femoral-facing surface 22 is precisely machined to interact with the femoral component 14 of the knee prosthesis 12. This is done at the factory so that the femoral-facing surface 22 of the spacer blank 30 and the femoral component 14 are precisely matched. The opposite flat surface 32 of the spacer blank 30 is unformed. The primary thickness T2 of the spacer blank 30 is equal to or thicker than the maximum spacer width that could be used during the knee arthroplasty procedure.

In a second step, the flat surface 32 of the spacer blank 30 is machined on-demand at the time of the knee arthroplasty. The connection features 28 are machined into the flat surface 32 of the spacer blank 30. In addition, the machining of the spacer blank 30 creates the primary thickness T1 for the spacer construct 18 that is precisely needed for a particular patient.

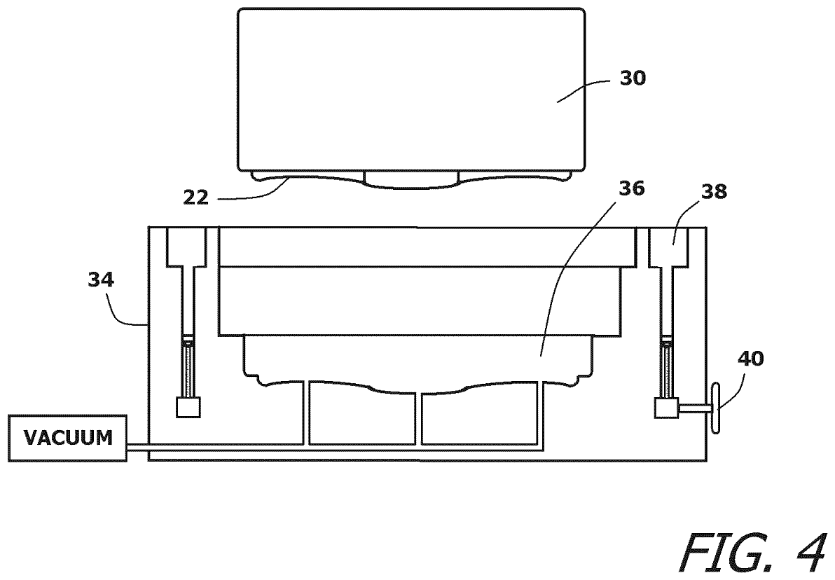

Referring to FIG. 4 in conjunction with FIG. 3, it will be understood that to machine the spacer blank 30 on-demand, the spacer blank 30 is first set into a retention base 34. The retention base 34 has a depression 36 that receives the femoral-facing surface 22 of the spacer blank 30. The spacer blank 30 is retained in the depression 36 using vacuum suction and/or mechanical clamps. The retention base 34 also has jig receptacles 38 that are capable of supporting various jigs. The jig receptacles 38 are adjustable in depth and are attached to a mechanical adjustment 40. The mechanical adjustment 40 enables the depth of the jig receptacles 38 to be selectively adjusted with great precision.

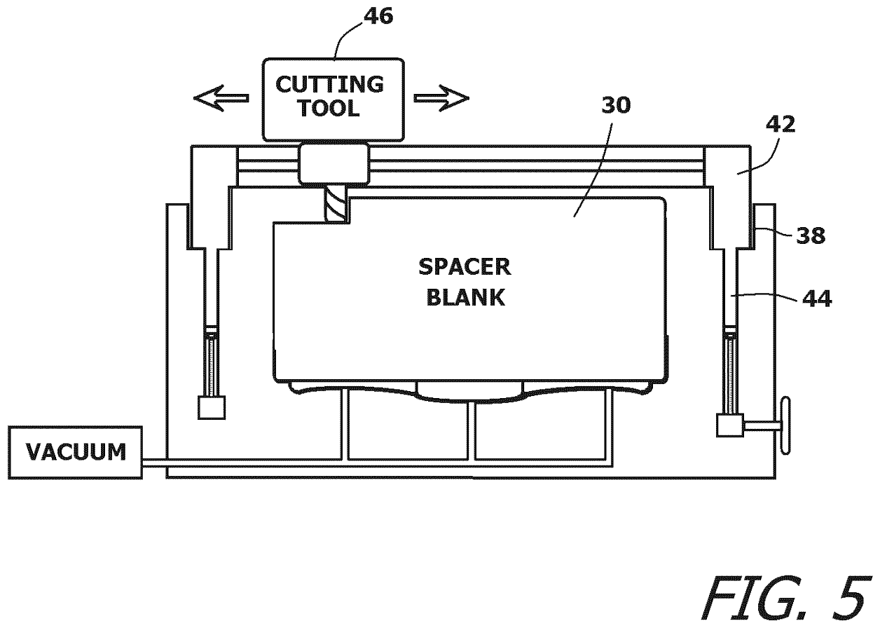

Referring to FIG. 5 in conjunction with FIG. 4, a cutting jig 42 is shown that is designed to interconnect with the retention base 34. The cutting jig 42 has supports 44 that extend into the jig receptacles 38 on the retention base 34. Accordingly, by adjusting the depth of the jig receptacles 38, the height of the cutting jig 42 relative to the spacer blank 30 can be selectively adjusted. The cutting jig 42 also has a cutting head 46 that is capable of cutting the spacer blank 30 in a flat plane at any selected height.

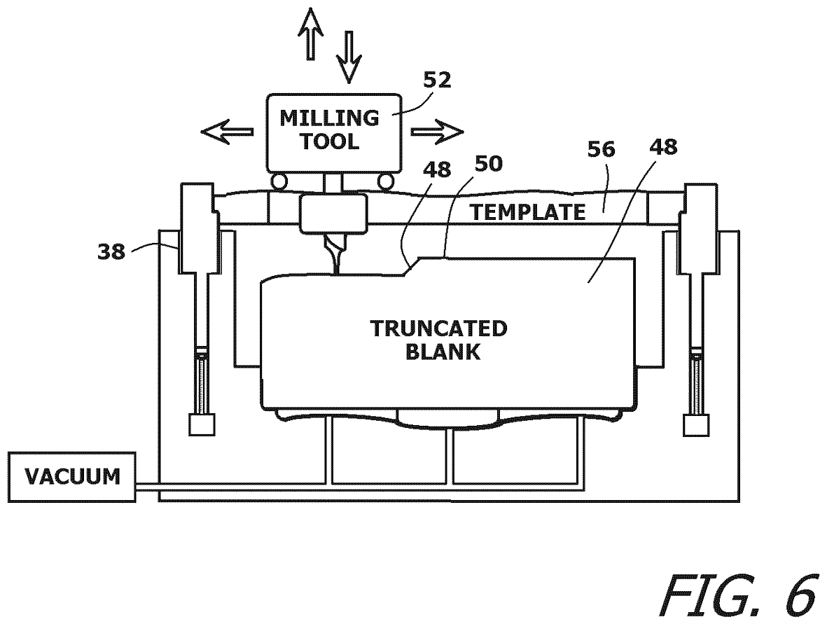

Referring to FIG. 6 in conjunction with FIG. 5, it can be seen that once the cutting jig 42 cuts the spacer blank 30, a truncated blank 48 is produced. The truncated blank 48 still has an unfinished top surface 50. The cutting jig 42 is removed and replaced with a milling jig 52. The milling jig 52 has supports 54 that extend into the jig receptacles 38 on the retention base 34. Accordingly, by adjusting the depth of the jig receptacles 38, the height of the milling jig 52 relative to the spacer blank 30 can be selectively adjusted. The milling jig 52 follows a pattern template 56 and cuts at least some of the connection features 28 into the truncated blank 48. Although one milling jig 52 is illustrated, it should be understood that multiple milling jigs can be used in sequence. As such, a first milling jig can cut part of the connection features 28, a second milling jig with a different cutting bit can cut other parts of the connection features 28, and yet another milling jig can be used to polish. The end result is the production of the final spacer construct 18.

Once the spacer construct 18 is finished being processed, the spacer construct 18 is custom sized to the exact needs of the knee arthroplasty. The custom spacer construct 18 is then attached to the tibial component 16 in the traditional manner. If there are any problems with fit, the spacer construct 18 can be reworked or a different spacer construct can be produced with slightly different settings to remove any problems encountered with fit.

It will be understood that the cutting jig 42 and the milling jigs 52 can be formed into an automated machine. In this manner, a surgeon can merely input the size needed and the machine will cut and mill the spacer blank 30 to the input dimensions. Alternatively, the present invention can also be practiced by using separate machines, wherein one machine cuts the spacer blank 30 to size and other machines cut the connector features 28 required by the tibial component 16 in use. There can also be a finishing machine (not shown) that polishes the cut surfaces to ensure the cut surfaces are smooth and free of any burrs.

In the embodiment of the invention described above, the tibial-facing surface 24 of the spacer construct 18 is formed on-demand. The on-demand production of the tibial-facing surface 24 is preferred. However, the same methodology can also be used to form the femoral-facing surface of the spacer construct 18. Forming the femoral-facing surface may be preferred when using knee joint prosthetic assemblies that do not utilize a detachable spacer construct. In some models of knee joint prosthetic assemblies, the spacer construct is fabricated as part of the tibial component of the prosthesis.

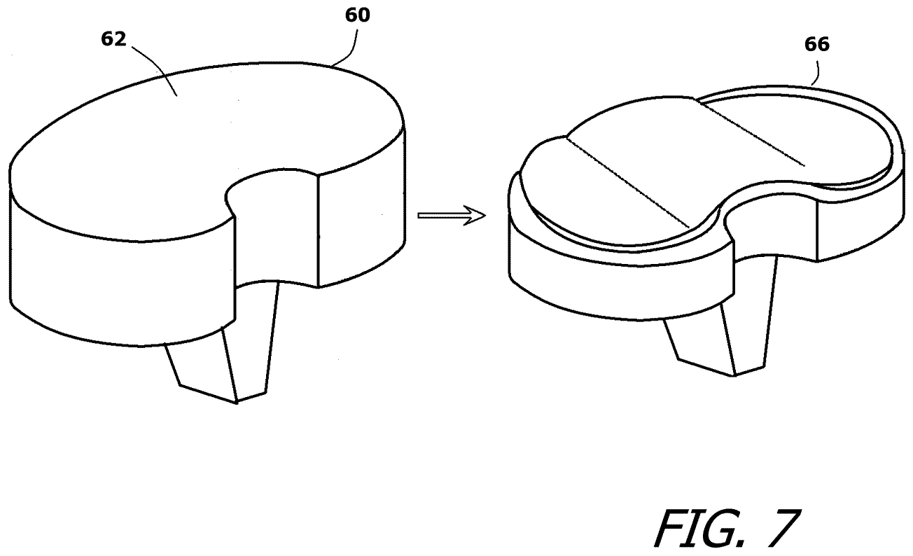

Referring to FIG. 7, it can be seen that a tibial blank 60 is provided. The tibial blank 60 is selected for the make and model of the knee prosthesis being used by the surgeon. The tibial blank 60 has a femoral-facing surface 62 that is unformed. The femoral-facing surface 62 is precisely machined on-demand to interact with the femoral component of the knee prosthesis. The features 64 on the femoral-facing surface are formed using a retention base and various jigs, as has previously been described. This produces a finished tibial component 66 that is customized to the needs of a particular patient.

It will be understood that the embodiments of the present invention that are illustrated and described are merely exemplary and that a person skilled in the art can make many variations to those embodiments. All such embodiments are intended to be included within the scope of the present invention as defined by the claims.

What is claimed is:

1. A method of customizing a knee prosthesis that has a femoral component, a tibial component, and a spacer construct, said method comprising the steps of;
installing said femoral component during a surgical procedure;
installing said tibial component during said surgical procedure;
measuring a gap space present in vivo between said femoral component and said tibial component;
providing a spacer blank;
shaping said spacer blank into said spacer construct during said surgical procedure, wherein said spacer construct is custom sized for use in said gap space; and
installing said spacer construct within said gap space.

2. The method according to claim 1, wherein providing a spacer blank includes providing said spacer blank with a first surface and an opposing second surface, wherein said first surface is pre-shaped to engage said femoral component.

3. The method according to claim 2, wherein shaping said spacer blank into said spacer construct includes machining said spacer blank at said second surface.

4. The method according to claim 2, wherein shaping said spacer blank includes cutting said shaper blank into a truncated blank and machining said truncated blank.

5. The method according to claim 2, wherein shaping said spacer blank includes placing said spacer blank in a jig and using forming tools that are guided by said jig to cut and shape said spacer blank.

6. The method according to claim 5, wherein said forming tools are selected from a group comprising a cutting tool, a milling tool, and a polishing tool.

7. The method according to claim 1, wherein providing said spacer blank includes providing said spacer blank with a first surface and an opposing second surface, wherein said second surface is pre-shaped to engage said tibial component.

8. The method according to claim 7, wherein shaping said spacer blank into said spacer construct includes machining said spacer blank at said first surface.

9. A method of customizing a spacer construct for use between a femoral component, and a tibial component of a knee prosthesis, wherein said spacer construct has machined features that face said femoral component and connection features that face said tibial component, said method comprising the steps of;
determining an optimal size for said spacer construct for use between said femoral component and said tibial component;
providing a spacer blank having said machined featured preformed thereon, wherein said spacer blank is larger than said optimal size;
providing a jig for receiving and retaining said spacer blank;
forming said connection features into said spacer blank while in said jig, therein creating said spacer construct.

10. The method according to claim 9, further including cutting said spacer blank to a shorter size, therein creating a truncated blank, wherein forming said connection features includes forming said connection features onto said truncated blank.

11. The method according to claim 9, wherein determining an optimal size for said spacer construct includes installing said femoral component into a patient during a surgical procedure;
installing said tibial component during said surgical procedure; and
measuring a gap space present in vivo between said femoral component and said tibial component, wherein said optimal size is optimal for said gap space.

12. The method according to claim 9, wherein forming said connection features into said spacer blank includes using tools on said spacer blank that are guided by said jig to cut and shape said connection features.

13. The method according to claim 12, wherein said tools are selected from a group comprising a cutting tool, a milling tool, and a polishing tool.

14. A method of customizing a spacer construct for use between a femoral component and a tibial component of a knee prosthesis, wherein said spacer construct has machined features, said method comprising the steps of:
determining an optimal size for said spacer construct for use between said femoral component and said tibial component;
providing a spacer blank, wherein said spacer blank is larger than said optimal size;
providing a jig for receiving and retaining said spacer blank;
forming said machined features into said spacer blank while in said jig, therein creating said spacer construct.

15. The method according to claim 14, further including cutting said spacer blank to a shorter size, therein creating a truncated blank, wherein forming said machined features includes forming said machined features onto said truncated blank.

16. The method according to claim 14, wherein determining an optimal size for said spacer construct includes installing said femoral component into a patient during a surgical procedure;
installing said tibial component during said surgical procedure; and
measuring a gap space present in vivo between said femoral component and said tibial component, wherein said optimal size is optimal for said gap space.

17. The method according to claim 14, wherein forming said machined features into said spacer blank includes using tools on said spacer blank that are guided by said jig to cut and shape said machined features.
